(19) United States
(12) Patent Application Publication
(10) Pub. No.: US 2024/0123102 A1
(43) Pub. Date: Apr. 18, 2024

(54) DISINFECTION ROBOT SYSTEM AND DISINFECTION METHOD USING SAME

(71) Applicant: CASE LAB CO., LTD, Suwon-si, Gyeonggi-do (KR)

(72) Inventors: Tae Yong KOOK, Hwaseong-si, Gyeonggi-do (KR); Eun Jin KIM, Suwon-si, Gyeonggi-do (KR)

(73) Assignee: CASE LAB CO., LTD, Suwon-si, Gyeonggi-do (KR)

(21) Appl. No.: 18/277,935

(22) PCT Filed: Sep. 17, 2021
(86) PCT No.: PCT/KR2021/012813

§ 371 Date: Aug. 18, 2023

(30) Foreign Application Priority Data
Feb. 19, 2021 (KR) 10-2021-0022846

(51) Int. Cl. A61L 2/10; A61L 2/24; A61L 9/20; G05D 1/648

(52) U.S. Cl. CPC A61L 2/10; A61L 2/24; A61L 9/20; G05D 1/648; A61L 2202/11; A61L 2202/16; G05D 2105/10

(57) ABSTRACT

A disinfection robot system, includes: a light disinfection unit having first UVC lamps exposed to the outside to perform disinfection through emission of ultraviolet light; a plurality of air suction and disinfection units each having a second UVC lamp, an air suction pipe adapted to locate the second UVC lamp therein and configured to prevent the ultraviolet light emitted from the second UVC lamp from being exposed to the outside, a plurality of photocatalyst-coated means disposed inside the air suction pipe to produce hydroxyl groups through the ultraviolet light emitted from the second UVC lamp, and a fan for sucking air to the interior of the air suction pipe; a sensing unit having a human presence sensor and a distance sensor; and a control unit for controlling the light disinfection unit and the air suction and disinfection units according to the information detected by the sensing unit.

### CROSS-REFERENCE TO PRIOR APPLICATIONS

This Application is a National Stage Patent Application of PCT International Application No. PCT/KR2021/012813 (filed on Sep. 17, 2021), which claims priority to Korean Patent Application No. 10-2021-0022846 (filed on Feb. 19, 2021), which are all hereby incorporated by reference in their entirety.

### BACKGROUND

The present invention relates to a disinfection robot system and a disinfection method using the same.

In multi-use facilities where a lot of humans exist, such as houses, hospitals, offices, and the like, it is necessary to periodically conduct sanitization or disinfection, and to do this, disinfection equipment is used. Conventional disinfection equipment makes use of ultraviolet-C (UVC) light emission or chemical injection to disinfect all kinds of airborne bacteria and viruses such as corona viruses.

However, since the UVC disinfection of the existing disinfection equipment is harmful for the human body, disadvantageously, it has to be conducted only in a space where no humans exist or in a night-time unmanned environment, and the chemical injection causes environmental problems because of the chemical remaining. Further, the existing disinfection equipment works by means of a worker's manual control, thereby making it difficult to manage the environments of such multi-use facilities and requiring labors for the management.

Because the disinfection equipment is a mobile robot, furthermore, it is necessary to enhance both of battery efficiency and disinfection effectiveness.

### SUMMARY

Accordingly, it is an object of the present invention to provide a disinfection robot system and a disinfection method using the same that are capable of solving environmental problems caused by the chemical remaining in a chemical injection method, performing immediate disinfection even in a space where humans coexist, without having any worker, and providing 24-hour disinfection function.

It is another object of the present invention to provide a disinfection robot system and a disinfection method using the same that are capable of minimizing the amount of power consumed of a built-in battery and maximizing disinfection effectiveness.

In order to achieve the above-mentioned object, a disinfection robot system according to an exemplary embodiment of the present invention includes a light disinfection unit having first UVC lamps exposed to the outside to perform disinfection through emission of ultraviolet light; a plurality of air suction and disinfection units each having a second UVC lamp, an air suction pipe adapted to locate the second UVC lamp therein and configured to prevent the ultraviolet light emitted from the second UVC lamp from being exposed to the outside, a plurality of photocatalyst-coated means disposed inside the air suction pipe to produce hydroxyl groups through the ultraviolet light emitted from the second UVC lamp, and a fan for sucking air to the interior of the air suction pipe; a sensing unit having a human presence sensor and a distance sensor; and a control unit for controlling the light disinfection unit and the air suction and disinfection units according to the information detected by the sensing unit.

The control unit may control output intensities of the first UVC lamps and the moving velocities of the first UVC lamps according to distance information detected by the distance sensor to thus control ultraviolet irradiation time.

The control unit may control the output intensities of the first UVC lamps so that the output intensities of the first UVC lamps are proportional to the square of a distance between the distance sensor and a disinfection object.

The control unit may receive human presence information from the human presence sensor to control lighting of the first UVC lamps.

The control unit may receive human presence information from the human presence sensor to control the rotational speeds of the fans so that the quantity of disinfected air of the air suction and disinfection units is adjusted.

The photocatalyst-coated means may be made by coating titanium dioxide onto the surfaces of solid bodies.

Each air suction and disinfection unit further may include a vortex generation structure disposed inside the air suction pipe to generate a vortex from the air sucked to the air suction pipe.

A disinfection method using the above-mentioned disinfection robot system according to another exemplary embodiment of the present invention includes the steps of: controlling output intensities of the first UVC lamps under the control of the control unit according to distance information detected by the distance sensor; and controlling the moving velocities of the first UVC lamps under the control of the control unit to thus control ultraviolet irradiation time.

A disinfection method using the above-mentioned disinfection robot system according to another exemplary embodiment of the present invention includes the steps of controlling output intensities of the first UVC lamps under the control of the control unit so that the output intensities of the first UVC lamps are proportional to the square of a distance between the distance sensor and a disinfection object.

A disinfection method using the above-mentioned disinfection robot system according to another exemplary embodiment of the present invention includes the steps of receiving human presence information from the human presence sensor to control lighting of the first UVC lamps under the control of the control unit.

A disinfection method using the above-mentioned disinfection robot system according to another exemplary embodiment of the present invention includes the steps of receiving human presence information from the human presence sensor and the distance sensor to control the rotational speeds of the fans under the control of the control unit so that the quantity of disinfected air of the air suction and disinfection units is adjusted.

According to the present invention, the disinfection robot system and the disinfection method using the same can solve the environmental problems caused by the chemical remaining in the existing chemical injection method. Further, the disinfection robot system and the disinfection method using the same can perform immediate disinfection even in a space where humans coexist, without having any worker, and provide 24-hour disinfection function. Moreover, the disinfection robot system and the disinfection method using the same can minimize the amount of power consumed of the built-in battery and maximize the disinfection effectiveness.

### BRIEF DESCRIPTION OF THE DRAWINGS

FIG. 1 is a block diagram showing a disinfection robot system according to the present invention.

FIG. 2 is a schematic perspective view showing the disinfection robot system according to the present invention.

FIG. 3 is an exemplary view showing an air suction and disinfection unit of the disinfection robot system according to the present invention.

FIG. 4 is another exemplary view showing the air suction and disinfection unit of the disinfection robot system according to the present invention.

### DETAILED DESCRIPTION

The terms used in the present invention are to be given their ordinary and customary meaning to a person of ordinary skill in the art, and are not to be limited to a special or customized meaning unless expressly so defined herein. It should be noted that the use of particular terminology when describing certain features or aspect of the disclosure should not be taken to imply that the terminology is being re-defined herein to be restricted to include any specific characteristics of the features or aspects of the disclosure with which that terminology is associated.

The present invention may be modified in various ways and may have several exemplary embodiments. Specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention.

Hereinafter, an explanation of a disinfection robot system according to an embodiment of the present invention will be given in detail with reference to the attached drawings so that the embodiment of the present invention may be carried out by those having ordinary skill in the art.

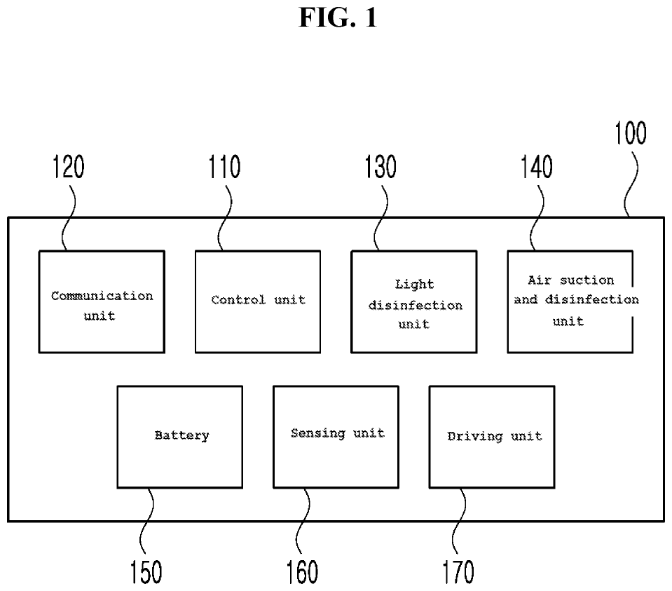

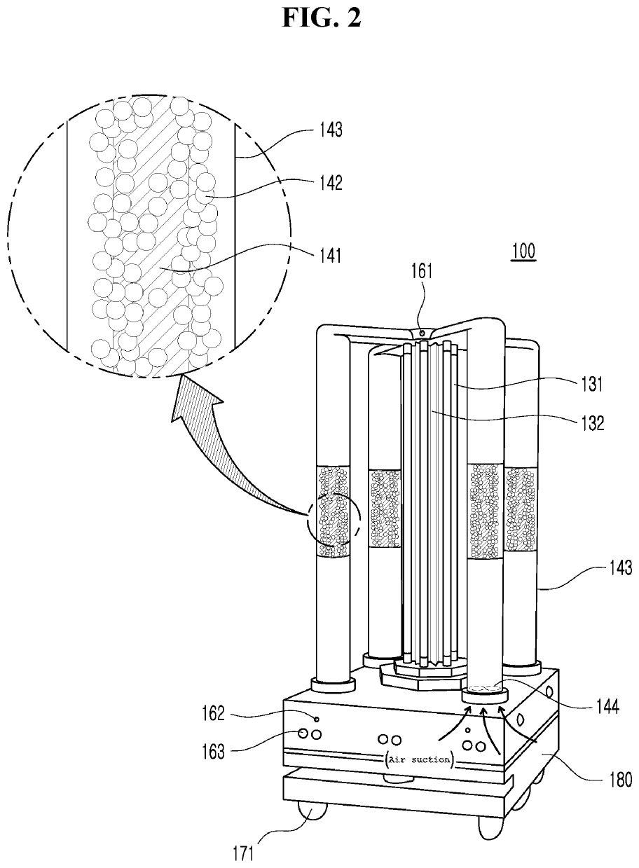

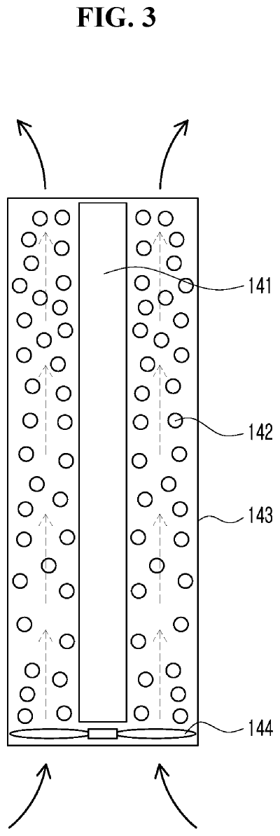

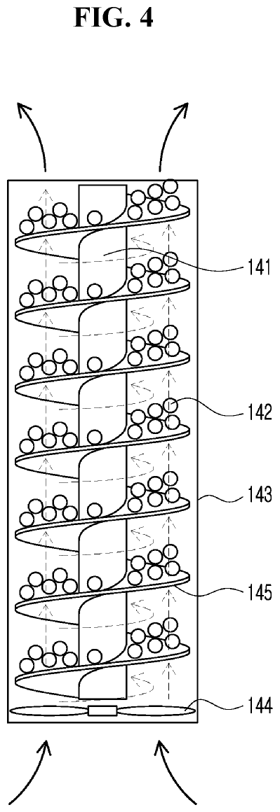

FIG. 1 is a block diagram showing a disinfection robot system according to the present invention, FIG. 2 is a schematic perspective view showing the disinfection robot system according to the present invention, FIG. 3 is an exemplary view showing an air suction and disinfection unit of the disinfection robot system according to the present invention, and FIG. 4 is another exemplary view showing the air suction and disinfection unit of the disinfection robot system according to the present invention.

As shown in FIGS. 1 to 3, a disinfection robot system 100 according to the present invention includes a control unit 110, a communication unit 120, a light disinfection unit 130, an air suction and disinfection unit 140, a battery 150, a sensing unit 160, and a driving unit 170.

The control unit 110 controls the communication unit 120, the light disinfection unit 130, the air suction and disinfection unit 140, the battery 150, the sensing unit 160, and the driving unit 170.

The communication unit 120 is accessed to an external device, such as a user terminal, a smartphone, a control tower/monitoring computer, and a server and thus transmits and receives data thereto and therefrom.

The sensing unit 160 includes a camera 161, a human presence sensor 162, a distance sensor 163 and receives external environment information required for operating the disinfection robot system 100. If necessary, the number of respective components of the sensing unit 160 is one or more.

The camera 161 is attached to top of a body 180 of the disinfection robot system 100, captures images around the disinfection robot system 100, and transmits the captured images to the control unit 110.

The human presence sensor 162 is a sensor for detecting the movement of an infrared light source, and since a human emits the infrared light of 8 to 14 μm from his or her body, the human presence sensor 162 detects human presence around the disinfection robot system 100 and thus transmits the detected information to the control unit 110.

The distance sensor 163 is an ultrasonic sensor and/or LiDAR sensor and thus attached to the body 180. The ultrasonic sensor emits ultrasonic waves, receives the ultrasonic waves reflected onto an external object, and detects a distance from the object or the movement of the object. The LiDAR sensor irradiates a laser beam onto an object and detects a distance from the object.

The driving unit 170 includes a motor and a mechanism that freely move the disinfection robot system 100 and further has a plurality of casters 171.

The light disinfection unit 130 includes a plurality of UVC lamps 131 and UVC reflection plates 132, and the UVC lamps 131 stand up on a central portion of the body 180 vertically in a longitudinal direction of the body 180 to emit UVC light in every direction. Further, the UVC reflection plates 132 reflect the UVC light emitted from the UVC lamps 131 so that the UVC light spreads outward well. Accordingly, the light disinfection unit 130 serves to neutralize or kill bacteria or viruses contained in the air or attached to an object around the disinfection robot system 100 and purify the pollutants in the air.

Basically, the disinfection work through the light disinfection unit 130 is done in a space where no humans exist. However, if the control unit 110 receives human presence information from the human presence sensor 162, the control unit 110 stops the operation of the light disinfection unit 130 immediately and thus prevents the UVC light from being exposed to the human.

The air suction and disinfection units 140 each have a UVC lamp 141, a plurality of photocatalyst-coated beads 142, an air suction pipe 143, and a fan 144.

The UVC lamp 141 is disposed long in a vertical direction inside the air suction pipe 143, and the plurality of photocatalyst-coated beads 142 are located around the UVC lamp 141 inside the air suction pipe 143. The fan 144 is located on the bottom of the air suction pipe 143, and as the fan 144 operates, the air suction pipe 143 sucks external air and exhausts the air to top thereof. If it is necessary to make the flow of air gentle, another fan may be mounted on top of the air suction pipe 143, and further, it does not have to locate the fan 144 on the bottom of the air suction pipe 143. The air suction pipe 143 has a structure shielded from UVC light to thus prevent the UVC light from being exposed to the outside thereof.

The photocatalyst-coated beads 142 are made by coating titanium dioxide (TiO₂) as a photocatalyst onto glass beads or synthetic resin beads, and if titanium dioxide receives ultraviolet light, it produces hydroxyl groups (—OH) having strong oxidizing power, oxidizes and decomposes organic compounds adsorbed thereto, and thus turns the organic compounds into water and carbonic acid gas. However, the titanium dioxide is not harmful for human bodies. Accordingly, the photocatalyst-coated beads 142 that receive the UVC light from the UVC lamp 141 serve to oxidize and decompose the pollutants such as bacteria, viruses, and the like contained in the air sucked to the interior of the air suction pipe 143 and thus perform disinfecting and antimicrobial actions. However, the photocatalyst-coated beads 142 do not have to have the shapes of beads, and for example, they may have different shapes such as the shapes of cubes. Further, the photocatalyst-coated beads 142 may be made of different materials excepting glass or synthetic resin.

The UVC lamp 141 emits the UVC light to allow the photocatalyst-coated beads 142 to conduct the disinfecting and antimicrobial actions and further eliminates the bacteria or viruses existing in the air introduced in the air suction pipe 143 through the UVC light emitted therefrom.

As shown in FIG. 3, the air sucked through the operation of the fan 144 moves upward in directions of arrows, and accordingly, the air from which the pollutants are eliminated through the disinfection action flows to top of the air suction pipe 143 and is then exhausted to the outside. Since the hydroxyl groups (—OH) produced in the air suction pipe 143 are contained in the exhausted air, the exhausted air can disinfect the environment around the disinfection robot system 100 and eliminate the pollutants contained therein.

If the fan 144 operates to allow external air to be sucked to the interior of the air suction pipe 143 under the control of the control unit 110, the UVC lamp 141 is turned on and emits UVC light so that the sucked air is disinfected directly with the UVC light and subjected to the disinfecting and antimicrobial treatments through the photocatalyst-coated beads 142. Accordingly, the air suction and disinfection units 140 serve to directly disinfect the microorganisms in the air through the UVC light and doubly disinfect them through the photocatalyst-coated beads 142, thereby optimizing the disinfection effectiveness.

Unlike the light disinfection unit 130 that operates only when no humans exist, the air suction and disinfection units 140 perform 24-hour disinfection consistently, irrespective of the existence of humans, because the air suction pipes 143 shield the UVC light emitted from the interior thereof.

As shown in FIG. 2, the disinfection robot system 100 has the plurality of air suction and disinfection units 140, and the air suction and disinfection units 140 stand up vertically on corners of top of the body 180. Because of the arrangements of the air suction and disinfection units 140 on the outside of the light disinfection unit 130, the UVC lamps 131 of the light disinfection unit 130 can be protected from the physical impacts applied from the outside.

Even though not shown, the body 180 is covered with an ultraviolet shielding cover adapted to prevent the UVC light emitted from the UVC lamps 131 of the light disinfection unit 130 from being transmitted to the outside, and further, an automatic shielding device is provided to automatically shield the UVC light emission.

Referring to FIG. 4, another example of the air suction and disinfection unit 140 is suggested, and in this case, the air suction and disinfection unit 140 further includes a vortex generation structure 145. The vortex generation structure 145 serves to generate a vortex from the air flowing therealong. As the flow of air becomes the vortex, the sucked air collides against the photocatalyst-coated beads 142 more actively to thus optimize the disinfection effectiveness. In FIG. 4, the vortex generation structure 145 has a spiral shape along the longitudinal direction of the UVC lamp 141 and the air suction pipe 143, but it may have various shapes, without being limited thereto.

The vortex generation structure 145 serves to support the photocatalyst-coated beads 142. If a large number of photocatalyst-coated beads 142 exist in the air suction pipe 143, air does not flow gently, and accordingly, the vortex generation structure 145 is disposed to allow the appropriate number of photocatalyst-coated beads 142 to be arranged thereon, while having appropriate distances from one another.

Further, the vortex generation structure 145 is a mesh structure in which the air flows well in the longitudinal direction of the air suction pipe 143. In specific, as shown by dotted arrows in FIG. 4, the sucked air through the fan 144 flows along the spiral shape of the vortex generation structure 145 and further passes through the vortex generation structure 145.

In the drawings, further, the air suction pipe 143 has the shape of a straight cylinder, but it may have a spiral shape so that the flow of air becomes a vortex. If necessary, further, an air suction portion and an air exhaust portion of the air suction pipe 143 may be different in size from each other, and the air suction pipe 143 may have a suction and exhaust path along which the vortex is generated well. As a result, the disinfection effectiveness becomes optimized.

The control unit 110 memorizes the map of a space in which the disinfection work is done, directly builds the map of the corresponding space, and recognizes the current position of the disinfection robot system 100 to control the disinfection work. Further, the control unit 110 has an algorithm for self-driving, and even though no separate work command is issued by a user, accordingly, the disinfection robot system 100 performs the disinfection work under the control of the control unit 110. Accordingly, the disinfection robot system 100 according to the present invention adopts the self-driving to thus perform the disinfection work, while ensuring 100% space coverage with no blind spot.

The control unit 110 receives the information of the space in which the disinfection robot system 100 is currently located and the distance value detected from the distance sensor 163 and adjusts the output values of the UVC lamps 131 of the light disinfection unit 130. In specific, since light energy such as ultraviolet light is based on a damped model inversely proportional to distance squared, the output values of the UVC lamps 131 are adjusted to allow about 70 mW/cm² as a disinfection threshold value of bacteria or viruses to be proportional to the square of a distance between the disinfection robot system 100 and a wall or object. Further, the moving speed of the disinfection robot system 100 is controlled according to the width of the space where the disinfection robot system 100 is located and the output values of the UVC lamps 131, thereby adjusting the UVC irradiation time of the UVC lamps 131, and if the disinfection object is too distant from the disinfection robot system 100 so that it is not within the disinfection threshold value, the moving speed of the disinfection robot system 100 becomes slow to extend the UVC irradiation time, thereby allowing the UVC light to be sufficiently irradiated onto the disinfection object.

For example, the intensity of electromagnetic waves from a light source P_s(W) according to a distance r is obtained by the following mathematical expression 1.

Intensity (W/cm²)=P_s/(4π×r²) [Mathematical expression 1]

Accordingly, the control unit 110 controls the outputs of the UVC lamps 131 that satisfy the disinfection threshold value through the following mathematical expression 2.

P_s=70(mW/Cm²)×(4π×r²) [Mathematical expression 2]

Like this, if the output intensities of the UVC lamps 131 and the UVC irradiation time are adjusted, the power consumption of the UVC lamps 131 is minimized to enhance the efficiency of the battery 150, while keeping the disinfection effectiveness of the disinfection robot system 100. In specific, there is no problem in maximizing the outputs of the UVC lamps 131 in a relatively large space, but in a relatively small space, it is efficient in terms of power consumption that the output intensities of the UVC lamps 131 and the UVC irradiation time are adjusted to satisfy only the disinfection threshold value corresponding to the small space.

The control unit 110 controls the fans 144 of the air suction and disinfection units 140 to adjust the air suction speeds introduced in the air suction pipes 143. If enough time for disinfection work is allowed, the introduced air stays long in the air suction pipes 143, thereby ensuring high disinfection effectiveness. Further, the control unit 110 receives the human presence information from the human presence sensor 162 and thus controls the rotational speeds of the fans 144 in consideration of a quantity of air purified and noise according to the detected result for the human presence. If humans exist around the disinfection robot system 100, the control unit 110 decreases the rotational speeds of the fans 144 to thus reduce the noise generated, and contrarily, if humans do not exist, the control unit 110 increases the rotational speeds of the fans 144.

Further, the disinfection robot system 100 according to the present invention is configured to allow an air inlet, an air outlet, and a fan installation position of the air suction pipe 143 to be adjusted in sectional area and to allow the rotational speed of the fan 144 inside the air suction pipe 143 to be adjusted, thereby controlling a quantity of air purified and generation of noise. Air quantity Q that is obtained by multiplying the sectional area Ain of the air inlet of the air suction pipe 143 by the velocity Vin of air coming through the air inlet is the same as the air quantity Q obtained by multiplying the sectional area A at which the fan 144 is installed by the velocity V of air passing through the fan and the air quantity Q obtained by multiplying the sectional area Aout of the air outlet of the air suction pipe 142 by the velocity Vout of air outgoing through the air outlet, so that the velocity V of air passing through the fan is calculated as (Vin+Vout)/2.

Accordingly, the sectional areas Ain, Aout, and A are adjustedly designed or the air velocity V is controlled, thereby controlling the quantity of air purified and the generation of noise. For example, if the sectional area Ain of the air inlet is larger than the sectional area A of the fan installation position and the sectional area Aout of the air outlet is larger than the sectional area A of the fan installation position, the velocity Vin of air coming and the velocity Vout of air outgoing become lower when compared with the velocity V of air passing through the fan, thereby reducing the quantity of air introduced and the quantity of noise generated.

The disinfection robot system 100 allows various data related to the disinfection treatment to be stored in an external server, and the external server integratedly controls the data related to the disinfection treatment and transmits control commands for the disinfection treatment to the disinfection robot system 100 to perform the disinfection work. If a plurality of disinfection robot systems 100 operate in a large area to perform disinfection works, the area is divided so that the disinfection robot systems 100 like swarm robots are controlled to thus perform the disinfection works.

The user accesses to the disinfection robot system 100 through the user terminal, smartphone, or control tower/monitoring computer, receives the information of the disinfection treatment from the disinfection robot system 100 in real time, and remotely controls the disinfection robot system 100.

The disinfection robot system 100 according to the present invention is configured to allow the light disinfection unit 130 to directly disinfect the area around the disinfection robot system 100 and to allow the air suction and disinfection units 140 to quickly disinfect the bacteria and viruses through air suction. In specific, the disinfection robot system 100 according to the present invention performs the disinfection work even in a space where humans exist, while preventing the ultraviolet light harmful for the human body from being exposed to the outside, so that the disinfection robot system 100 can always operate even in the space where humans exist and perform air purification and air pollution prevention even in an indoor space at which air ventilation is hard.

Unlike the existing system that operates only in a space where no humans exist, the disinfection robot system 100 according to the present invention performs the disinfection work immediately, even in a space where humans co-exist. That is, the existing system operates at night in an environment where no humans exist after working time in hospitals, houses, offices, working places, and the like, but the disinfection robot system 100 according to the present invention always performs 24-hour disinfection work, even during working time, thereby optimizing the conveniences and effectiveness of the disinfection work. Further, the disinfection robot system 100 according to the present invention solves the problems occurring in the existing method where disinfection is performed with the direct exposure of ultraviolet light to thus cause discoloring of furniture and destruction of plant cells if even the space where no humans exist is exposed to the ultraviolet light for long hours.

The present invention may be modified in various ways and may have several exemplary embodiments. Specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention. While this invention is illustrated and described in a preferred embodiment, the device may be produced in many different configurations, forms, and materials. There is depicted in the drawings, and will hereinafter be described in detail, a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the present invention.

The disinfection robot system and the disinfection using the same according to the present invention are applicable to multi-use facilities where a lot of humans exist, such as houses, hospitals, offices, and the like to perform sanitization or disinfection.

1. A disinfection robot system comprising:
a light disinfection unit having first UVC lamps exposed to the outside to perform disinfection through emission of ultraviolet light;
a plurality of air suction and disinfection units each having a second UVC lamp, an air suction pipe adapted to locate the second UVC lamp therein and configured to prevent the ultraviolet light emitted from the second UVC lamp from being exposed to the outside, a plurality of photocatalyst-coated means disposed inside the air suction pipe to produce hydroxyl groups through the ultraviolet light emitted from the second UVC lamp, and a fan for sucking air to the interior of the air suction pipe;
a sensing unit having a human presence sensor and a distance sensor; and
a control unit for controlling the light disinfection unit and the air suction and disinfection units according to the information detected by the sensing unit.

2. The disinfection robot system according to claim 1, wherein the control unit controls output intensities of the first UVC lamps and the moving velocities of the first UVC lamps according to distance information detected by the distance sensor to thus control ultraviolet irradiation time.

3. The disinfection robot system according to claim 1, wherein the control unit controls the output intensities of the first UVC lamps so that the output intensities of the first UVC lamps are proportional to the square of a distance between the distance sensor and a disinfection object.

4. The disinfection robot system according to claim 1, wherein the control unit receives human presence information from the human presence sensor to control lighting of the first UVC lamps.

5. The disinfection robot system according to claim 1, wherein the control unit receives human presence information from the human presence sensor to control the rotational speeds of the fans so that the quantity of disinfected air of the air suction and disinfection units is adjusted.

6. The disinfection robot system according to claim 1, wherein the photocatalyst-coated means are made by coating titanium dioxide onto the surfaces of solid bodies.

7. The disinfection robot system according to claim 1, wherein each air suction and disinfection unit further comprises a vortex generation structure disposed inside the air suction pipe to generate a vortex from the air sucked to the air suction pipe.

8. A disinfection method using the disinfection robot system according to claim 1, the method comprising the steps of:
controlling output intensities of the first UVC lamps under the control of the control unit according to distance information detected by the distance sensor; and
controlling the moving velocities of the first UVC lamps under the control of the control unit to thus control ultraviolet irradiation time.

9. A disinfection method using the disinfection robot system according to claim 1, the method comprising the step of controlling output intensities of the first UVC lamps under the control of the control unit so that the output intensities of the first UVC lamps are proportional to the square of a distance between the distance sensor and a disinfection object.

10. A disinfection method using the disinfection robot system according to claim 1, the method comprising the step of receiving human presence information from the human presence sensor to control lighting of the first UVC lamps under the control of the control unit.

11. A disinfection method using the disinfection robot system according to claim 1, the method comprising the step of receiving human presence information from the human presence sensor and the distance sensor to control the rotational speeds of the fans under the control of the control unit so that the quantity of disinfected air of the air suction and disinfection units is adjusted.
